(19) United States
(12) Patent Application Publication
(10) Pub. No.: US 2024/0025940 A1
(43) Pub. Date: Jan. 25, 2024

(54) NOVEL ESTERS OF MEDROXYPROGESTERONE

(71) Applicants: Kalak Abdul HAFEEZ, Bengaluru (IN); Sandhya PITTALA, Hyderabad (IN)

(72) Inventors: Kalak Abdul HAFEEZ, Bengaluru (IN); Sandhya PITTALA, Hyderabad (IN)

(21) Appl. No.: 17/542,540

(22) PCT Filed: Jul. 18, 2019
(86) PCT No.: PCT/IB2019/056136

§ 371 Date: Dec. 6, 2021

(30) Foreign Application Priority Data
Jun. 5, 2021 (IN) 201941022324

(51) Int. Cl. C07J 1/00

(52) U.S. Cl. CPC C07J 1/0033

(57) ABSTRACT

The present disclosure relates to novel ester forms of medroxyprogesterone process for their preparation and pharmaceutical compositions comprising the same. The present disclosure also relates to use of the novel ester forms in hormonal therapies such as, for example, contraception, hormone replacement therapy, endometriosis, etc.

### FIELD OF THE INVENTION

The present disclosure relates to technical field of pharmaceutical. In particular, the present disclosure relates to novel esters of medroxyprogesterone useful as hormonal therapeutic agents, methods for their preparation, and pharmaceutical compositions comprising the same.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Medroxyprogesterone is chemically known as (6S,8R,9S,1 OR,13S 4S,17R)-17-acetyl-17-hydroxy-6,10,13-trimethyl-2,6,7,8,9,11,12,14,15,16-decahydro-1H- cyclopenta[a]phenanthren-3-one, and is structurally represented by the following formula I. U.S. Pat. No. 3,377,364 discloses medroxyprogesterone and its preparation method.

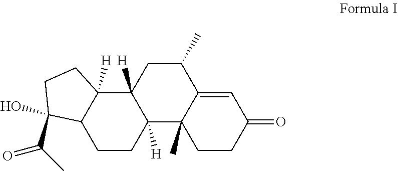 [SMILES: CC(=O)[C@@]1(O)CC[C@@H]2C1CC[C@H]1[C@H]2C[C@H](C)C2=CC(=O)CC[C@@]21C]

Medroxyprogesterone acetate, commonly abbreviated as MPA, is a hormonal medication of the progestin type. It is used as a contraceptive and as a part of menopausal hormone therapy. it is also used to treat endometriosis, abnormal uterine bleeding, abnormal sexuality in males, and certain types of cancer, among other indications. Medroxyprogesterone acetate and its preparation method are described in U.S. Pat. No. 3,377,364 and US Publication No. 2009/0012321 A1, Medroxyprogesterone acetate (MPA) is generically known as 6α-methyl-17α-hydroxyprogesterone acetate. Medroxyprogesterone acetate has the chemical name [(6S,8R,9S,10R,13S,14S,17R)-17-acetyl-6,10,13-trimethyl-3-oxo-2,6 ,8,9,11,12,14,15,16-decahydro-1H-cyclopenta[a]phenanthren-17-yl] acetate, and is structurally represented by the following formula II.

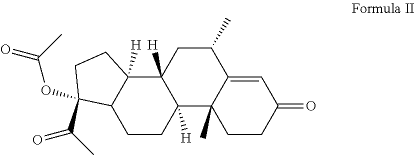 [SMILES: CC(=O)O[C@]1(C(C)=O)CC[C@@H]2C1CC[C@H]1[C@H]2C[C@H](C)C2=CC(=O)CC[C@@]21C]

U.S. Pat. No. 3,377,364 describes the synthesis of medroxyprogesterone acetate, pharmaceutical formulations of medroxyprogesterone acetate, and also discloses the therapeutic use of this molecule as oral and parenteral progestational agent.

Although medroxyprogesterone acetate is therapeutically beneficial, there still exists a need in the art for novel forms of medroxyprogesterone which have beneficial pharmaceutical applications and can be used as alternatives to the progestational agents which are currently available. The inventor has how surprisingly and unexpectedly found novel forms of medroxyprogesterone which have desirable properties such as, for example, improved bioavailability and pharmacokinetics, advantageous formulation properties, good stability and patient compliance. As such, the novel forms are pharmaceutically acceptable and can be used to prepare pharmaceutical formulations.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that provided herein applies and the definition of that term in the reference does not apply.

Groupings of alternative elements or embodiments of the invention disclosed herein are not to be construed as limitations. Each group member can he referred to and claimed individually or in any combination with other members of the group or other elements found herein. One or more members of a group can be included in, or deleted from, a group for reasons of convenience and/or patentability.

### OBJECTS OF THE INVENTION

It is an object of the present disclosure to provide novel forms of medroxyprogesterone suitable for use as hormonal therapy drugs.

It is another object of the present disclosure to provide novel forms of medroxyprogesterone having improved bioavailability.

It is another object of the present disclosure to provide novel forms of medroxyprogesterone having enhanced pharmacokinetic properties.

It is another object of the present disclosure to provide novel forms of medroxyprogesterone having good physical and chemical stability.

It is another object of the present disclosure to provide novel forms of medroxyprogesterone that have advantageous formulation properties.

### SUMMARY

Aspects of the present disclosure relate to novel ester forms of 6α-methyl-hydroxyprogesterone (medroxyprogesterone) suitable for use as hormonal therapy drugs.

In one aspect, the present disclosure is directed to 6α-methyl-17α-hydroxyprogesterone propiolate (also referred to herein as medroxyprogesterone 17-propiolate) having formula Ia.

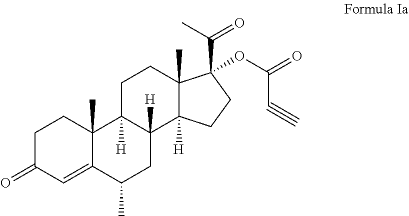

In another aspect, the present disclosure is directed to 6α-methyl-17α-hydroxyprogesterone cyclopropanecarboxylate (also referred to herein as medroxyprogesterone 17-cyclopropanecarboxylate) having formula Ib.

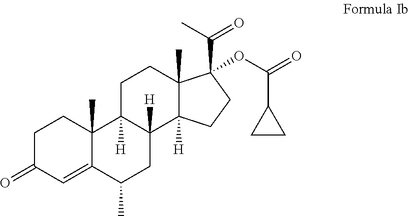

in another aspect, the present disclosure is directed to 6α-methyl-17α-hydroxyprogesterone isovalerate (also referred to herein as medroxyprogesterone t7-isovalerate having formula Ic.

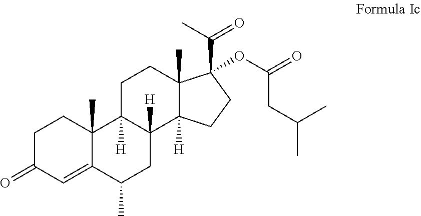

In another aspect, the present disclosure is directed to 6α-methyl-17α-hydroxyprogesterone cyclopentylacetate (also referred to herein as medroxyprogesterone 17-cyclopentylacetate) having formula Id.

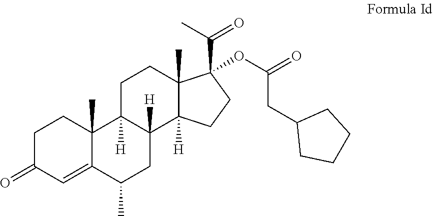

In another aspect, the present disclosure is directed to 6α-methyl-17α-hydroxyprogesterone cyclopropanepropanoate (also referred to herein as medroxyprogesterone 17-cyclopropanepropanoate) having formula Ie.

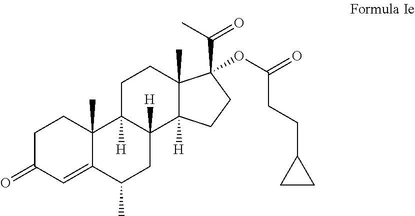

In another aspect, the present disclosure is directed to 6α-methyl-17α-hydroxyprogesterone isobutyrate (also referred to herein as medroxyprogesterone 17-isobutyrate) having formula If.

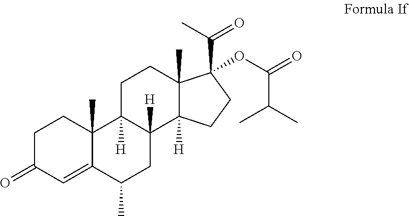

According to embodiments of the present disclosure, each of the above mentioned ester forms of medroxyprogesterone can be used as a hormonal therapeutic agent. The ester forms of medroxyprogesterone disclosed herein can exhibit desirable properties such as, for example, improved physical and chemical stability, improved bioavailability, enhanced pharmacokinetic properties, and advantageous formulation properties.

Another aspect of the present disclosure is directed to a method of providing contraception and of treating hormone-dependent conditions, the method comprising administering, to a mammal in need thereof, a therapeutically effective amount of a medroxyprogesterone ester, which is a compound selected from the group consisting of medroxyprogesterone 17--propiolate, medroxyprogesterone 17-cyclopropanecarboxylate, medroxyprogesterone 17-isovalerate, medroxyprogesterone 17-cyclopentylacetate, medroxyprogesterone 17-cyclopropanepropanoate, medroxyprogesterone 17-isobutyrate and a pharmaceutically acceptable salt thereof, or a pharmaceutical composition comprising such an ester or a pharmaceutically acceptable salt thereof.

Various objects, features, aspects and advantages of the inventive subject matter will become more apparent from the following detailed description of preferred embodiments.

### BRIEF DESCRIPTION OF THE DRAWINGS

FIG. 1 shows a high resolution mass spectrometry (HRMS) spectrum of medroxyprogesterone.

FIG. 2 shows a ¹H-nuclear magnetic resonance (NMR) spectrum of medroxyprogesterone.

FIG. 3 shows a FIRMS spectrum of medroxyprogesterone 17-propiolate prepared in accordance with embodiments of the present disclosure.

FIG. 4 shows a HRMS spectrum of medroxyprogesterone cyclopropanecarboxylate prepared in accordance with embodiments of the present disclosure.

FIG. 5 shows a HRMS spectrum of medroxyprogesterone 17-isovalerate prepared in accordance with embodiments of the present disclosure.

### DETAILED DESCRIPTION OF THE INVENTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment s included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, process conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms are used herein. To the extent a term used in a claim is not defined below, it should be given the broadest definition persons in the pertinent art have given that term as reflected in printed publications and issued patents at the time of filing.

The present disclosure provides ester forms of 6α-methyl-17α-hydroxyprogesterone (medroxyprogesterone) which are suitable for use as hormonal therapy drugs. The disclosed medroxyprogesterone esters are pharmaceutically acceptable and can he used to prepare pharmaceutical formulations. Further, the medroxyprogesterone esters disclosed herein can exhibit desirable properties such as, for example, improved physical and chemical stability, improved bioavailability, enhanced pharmacokinetic properties, and advantageous formulation properties.

In one aspect, the present disclosure is directed to 6a-methyl-17α-hydroxyprogesterone propiolate (also referred to herein as medroxyprogesterone 17-propiolate) having formula Ia.

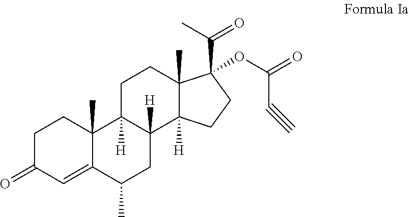

In another aspect, the present disclosure is directed to 6α-methyl-17α-hydroxyprogesterone cyclopropanecarboxylate (also referred to herein as medroxyprogesterone 17-cyclopropanecarboxylate) having formula Ib.

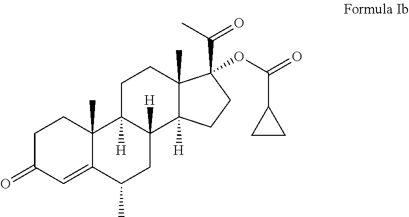

in another aspect, the present disclosure is directed to 6α-methyl-17α-hydroxyprogesterone isovalerate (also referred to herein as medroxyprogesterone t7-isovalerate having formula Ic.

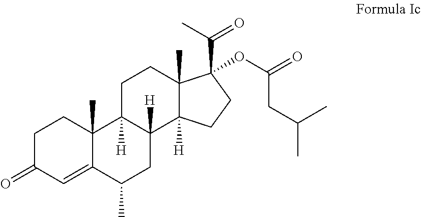

In another aspect, the present disclosure is directed to 6α-methyl-17α-hydroxyprogesterone cyclopentylacetate (also referred to herein as medroxyprogesterone cyclopentylacetate) having formula Id.

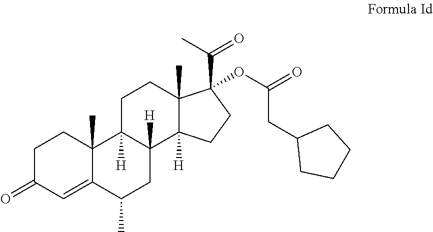

In another aspect, the present disclosure is directed to 6α-methyl-17α-hydroxyprogesterone cyclopropanepropanoate (also referred to herein as medroxyprogesterone 17-cyclopropanepropanoate) having formula Ie.

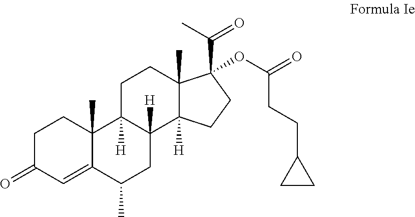

In another aspect, the present disclosure is directed to 6α-methyl-17α-hydroxyprogesterone isobutyrate (also referred to herein as medroxyprogesterone 17-isobutyrate) having formula If.

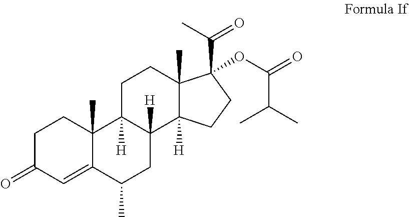

In another aspect, the present disclosure provides a pharmaceutical composition comprising a medroxyprogesterone ester, which is a compound selected from:

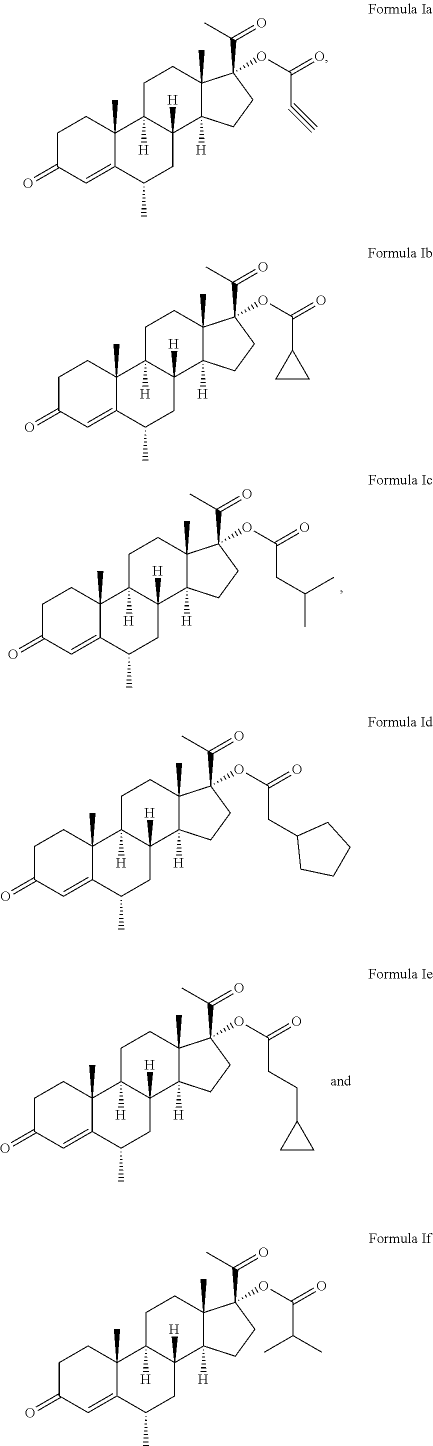

or a pharmaceutically acceptable salt thereof, and at least one pharmaceutically acceptable carrier,

Another aspect of the present disclosure is directed to a method of providing contraception and of treating hormone-dependent conditions, the method comprising administering, to a mammal in need thereof, a therapeutically effective amount of a medroxyprogesterone ester, which is a compound selected from the group consisting of:

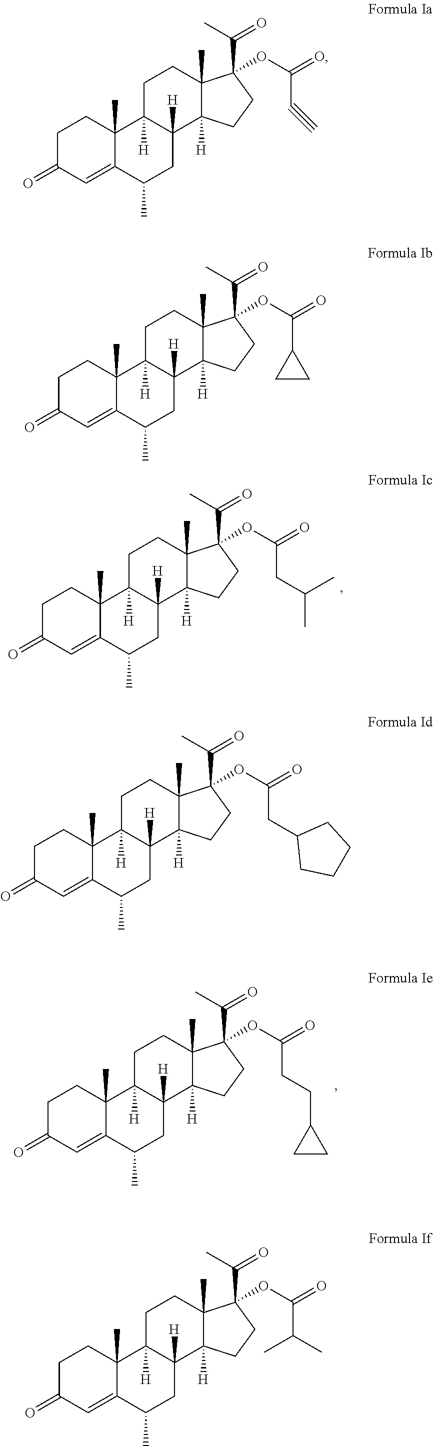

and a pharmaceutically acceptable salt thereof,
or a pharmaceutical composition comprising such an ester or a pharmaceutically acceptable salt thereof.

In a particular embodiment, the disclosed medroxyprogesterone ester of formula Ia, Ib, Ic, Id, Ie or If can be therapeutically used to induce contraception in a mammal. In this embodiment, a therapeutically effective amount of the medroxyprogesterone ester can be administered to said mammal via parenteral route for the purpose of achieving contraceptive effect in said mammal. The parenteral route includes intravenous, intra-arterial, intramuscular, subcutaneous, transdermal, intradermal and intraperitoneal administration. Preferably, the medroxyprogesterone ester is administered intramuscularly or subcutaneously.

In one embodiment, the mammal may be a male or female human. In a particular embodiment, the mammal is a human female of reproductive age.

While it is possible for the medroxyprogesterone esters to be administered alone, it is preferable to present them as a pharmaceutical formulation. The formulations for human use comprise at least one medroxyprogesterone ester, as defined above, together with one or more pharmaceutically acceptable carriers and optionally other therapeutic ingredients.

In various embodiments, the disclosed medroxyprogesterone esters are formulated in a pharmaceutically acceptable carrier. Suitable pharmaceutically acceptable carriers for parenteral administration include, but not limited to, diluents, preservatives, plasticizers, wetting agents, emulsifiers, dispersants, lubricants, solvents, release modifying agents, binders, antioxidants, surfactants, stabilizing agents, tonicity adjusting agents and buffers. The formulations may conveniently be presented in unit dosage form and may be prepared by any methods well known in the art of pharmacy. The amount of the medroxyprogesterone ester that may be combined with a carrier to produce a single dose may vary depending upon the subject being treated, and the particular mode of administration. In various embodiments, the pharmaceutical composition comprises the medroxyprogesterone ester at a concentration ranging from about 0.001 to about 90% by weight of the composition.

In one embodiment of this aspect, hormone-dependent conditions that may be treated by the medroxyprogesterone ester of the present disclosure can include, but not limited to, hormone replacement therapy, endometriosis, leiomyoma, abnormal uterine bleeding, abnormal sexuality in male, premenstrual syndrome and hormonal dependent cancers.

As used herein, the term “therapeutically effective amount” describes a quantity of medroxyprogesterone ester sufficient to achieve a desired effect in a mammal being treated with that ester. The therapeutically effective amount may vary depending upon a variety of factors including, particular state or condition being treated, age, weight, general health, and responsiveness of a mammal to be treated.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Example 1: Hydrolysis of medroxyprogesterone Acetate into medroxyprogesterone

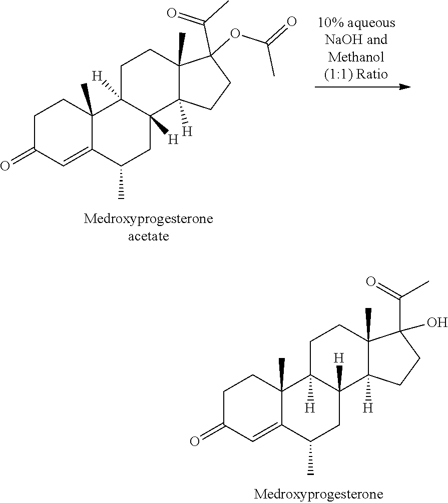

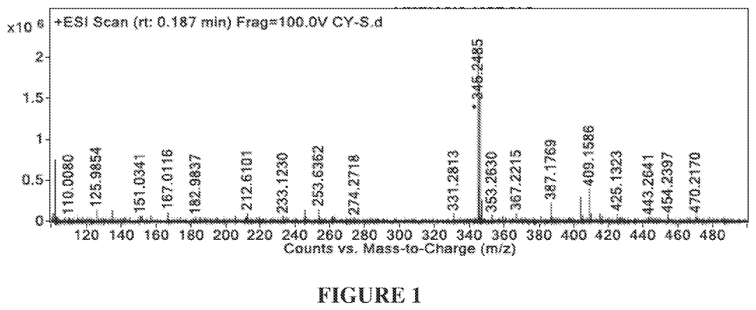

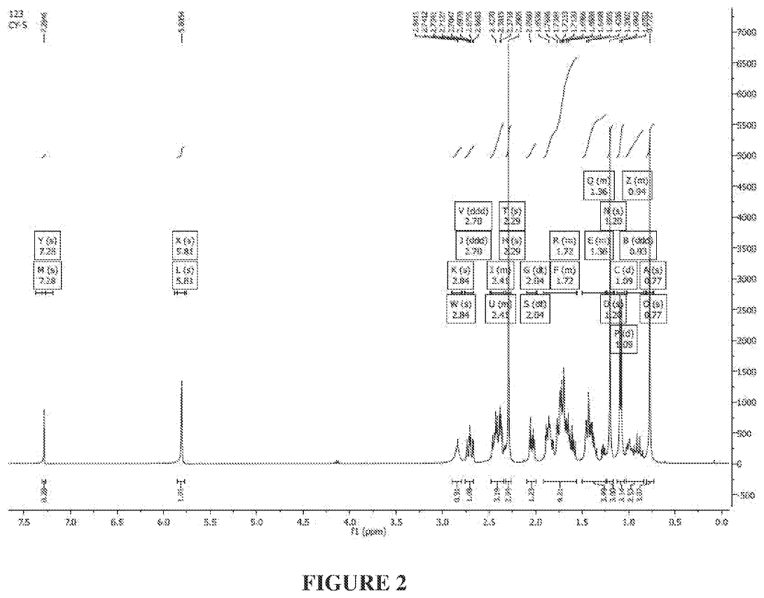

To the stirred solution of medroxyprogesterone acetate (5 g) in methanol (50 ml), 10 aqueous sodium hydroxide solution was added (50 ml), and the reaction was refluxed. After completion of reaction as per TLC (after 5 h), methanol was evaporated, 100 ml of distilled water was added to the reaction mixture and its pH was made neutral slightly with 6N HCl. Further, aqueous portion was again extracted twice with ethyl acetate (200 m1). Combined ethyl acetate layer was first washed with ice cold water (400 ml), and then with brine water (400 ml). Ethyl acetate layer was dried over anhydrous sodium sulphate and finally evaporated on the rotary evaporator to afford medroxyprogesterone in 93% yield. Purified compound was characterized by high resolution mass spectrometry (HRMS) and nuclear magnetic resonance (NMR). The HRMS and ¹H-NMR spectra of thus obtained medroxyprogesterone are shown in FIGS. 1 and 2 respectively. The mass spectral data were as follows:

| m/z | z | Abund |
|---|---|---|
| 61.0072 | | 159674.28 |
| 64.0155 | | 526888.12 |
| 73.5315 | 2 | 167475.46 |
| 74.0597 | | 196464.07 |
| 81.5203 | | 250526.92 |
| 102.1272 | 1 | 775484.67 |
| 125.9854 | | 143819.93 |
| 135.1006 | | 136246.92 |
| 167.0116 | | 115285.29 |
| 212.6101 | | 107472.24 |
| 245.6476 | 2 | 145626.09 |
| 253.6362 | 2 | 156509.65 |
| 345.2485 | | 2058626.12 |
| 345.2931 | | 386783.85 |
| 345.3128 | | 474082.23 |
| 345.341 | 1 | 425906.26 |
| 345.3618 | | 221438.06 |
| 346.2438 | 1 | 1571042.98 |
| 346.2797 | | 167221.23 |
| 347.246 | 1 | 262691.71 |
| 367.2215 | | 107881.6 |
| 387.1769 | 1 | 235925.85 |
| 404.2032 | 1 | 302490.86 |

### Example 2: Synthesis of medroxyprogesterone 17-propiolate

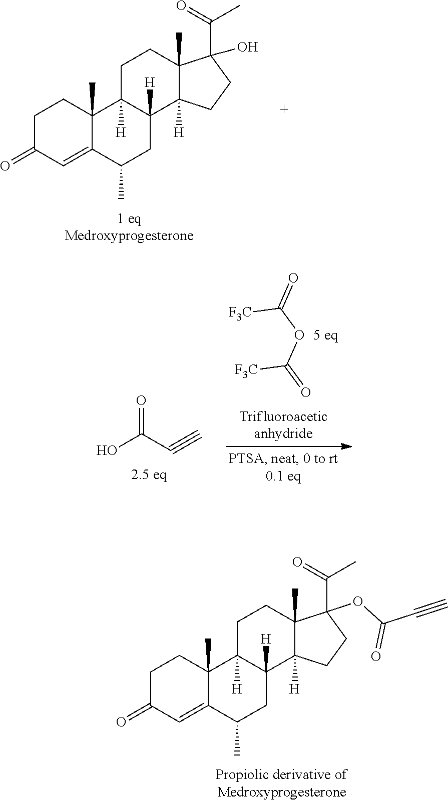

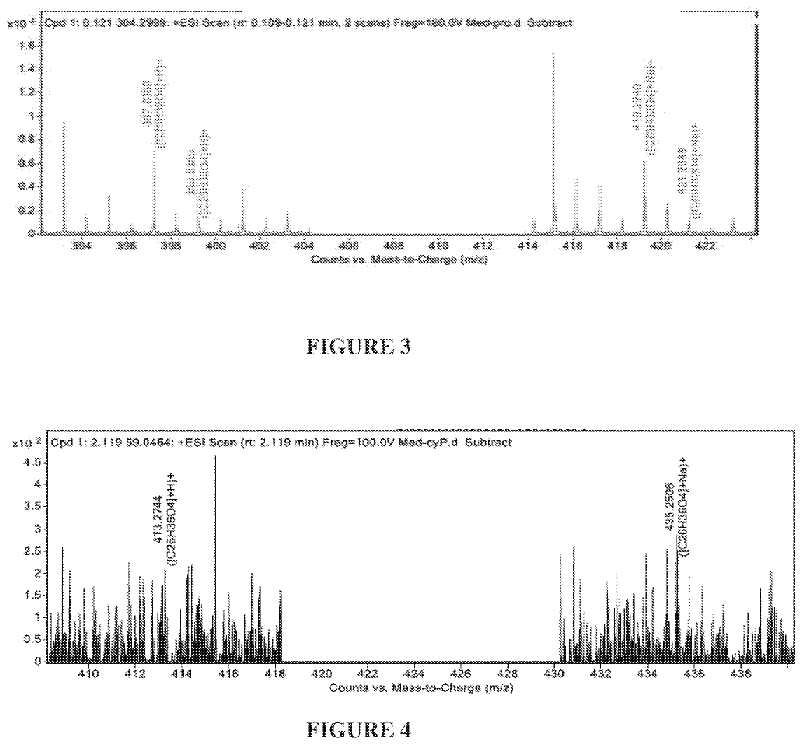

Propiolic acid 3.6 mmol (0.254 ml) and trifluoroacetic anhydride 7.2 mmol (1.46 nil) were taken in round bottom flask and stirred under ice cooled condition. To the reaction mixture 0.5 g of medroxyprogesterone was added in portions and then catalytic amount of p-toluenesulfonic acid (PTSA) was added (around 2 mg). The reaction was stirred at room temperature and its progress was monitored via TLC using ethyl acetate and hexane (40:60 ratio) as solvent system. After completion of the reaction, 50 ml of saturated sodium bicarbonate was added to the reaction mixture and twice extracted with ethyl acetate (50×2 ml). Combined ethyl acetate layer was first washed with ice cold water (200 ml), and then with brine water (100 ml). Ethyl acetate layer was dried over anhydrous sodium sulphate and finally evaporated on rotary evaporator to afford medroxyprogesterone 17-propiolate compound. The HRMS spectrum of the medroxyprogesterone 17-propiolate is shown in FIG. 3. The mass spectral data were as follows:

| m/z | Calc m/z | Diff(ppm) | z | Abund | Formula | Ion |
|---|---|---|---|---|---|---|
| 397.2359 | 397.2373 | 3.69 | 1 | 7102.32 | C25H32O4 | (M + H)+ |
| 398.2392 | 398.2407 | 3.9 | 1 | 1850.38 | C25H32O4 | (M + H)+ |
| 399.2399 | 399.2437 | 9.36 | 1 | 1208.57 | C25H32O4 | (M + H)+ |
| 419.224 | 419.2193 | −11.33 | 1 | 6549.71 | C25H32O4 | (M + Na)+ |
| 420.2269 | 420.2227 | −10.14 | 1 | 2133.63 | C25H32O4 | (M + Na)+ |
| 421.2348 | 421.2256 | −21.86 | 1 | 1106.69 | C25H32O4 | (M + Na)+ |

### Example 3: Synthesis of medroxyprogesterone 17-cyclopropanecarboxylate

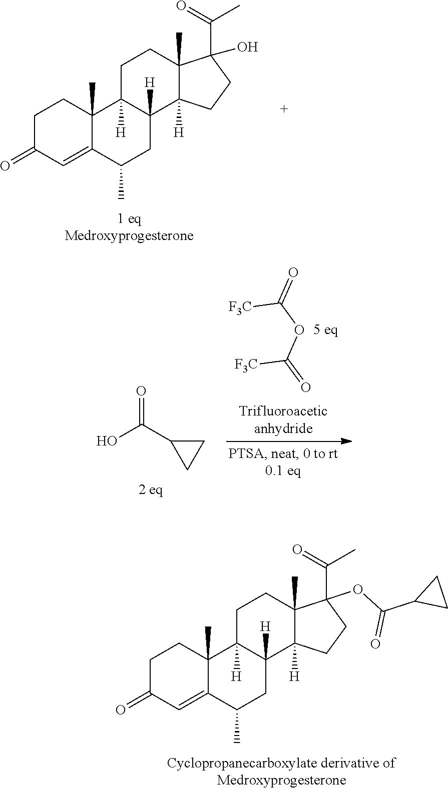

In round bottom flask cyclopropyl carboxylic acid 2.8 mmol (0.24 ml) and trifluoroacetic anhydride 7.2 mmol (L46 ml) were taken and stirred under ice cooled condition. To the reaction mixture 0.5 g of medroxyprogesterone was added in portions and then catalytic amount of p-toluenesulfonic acid (PTSA) was added (around 2 mg). The reaction mixture was stirred at room temperature and its progress was monitored via TLC using ethyl acetate and hexane (30:70 ratio) as solvent system. After 6 hours around 75 to 85 percent conversion took place. The reaction mixture was further stirred for 2 hours, and after that 50 ml of saturated sodium bicarbonate was added to the reaction mixture and extracted with ethyl acetate (100 ml). Combined ethyl acetate layer was first washed with ice cold water (200 ml), and then with brine water (100 ml). Ethyl acetate layer was dried over anhydrous sodium sulphate and finally evaporated on rotary evaporator to afford medroxyprogesterone 17-cyclopropanecarboxylate compound. The HRMS spectrum of the medroxyprogesterone 17-cyclopropanecarboxylate is shown in FIG. 4. The mass spectral data were as follows:

| m/z | Calc m/z | Diff(ppm) | z | Abund | Formula | Ion |
|---|---|---|---|---|---|---|
| 413.2744 | 413.2686 | −13.88 | 1 | 212.33 | C26H36O4 | (M + H)+ |
| 435.2506 | 435.2506 | −0.04 | 1 | 303.42 | C26H36O4 | (M + Na)+ |

### Example 4: Synthesis of medroxyprogesterone 17-isovalerate

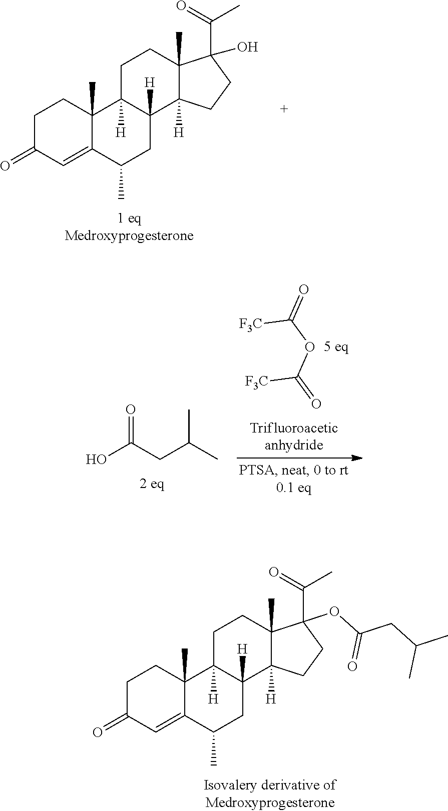

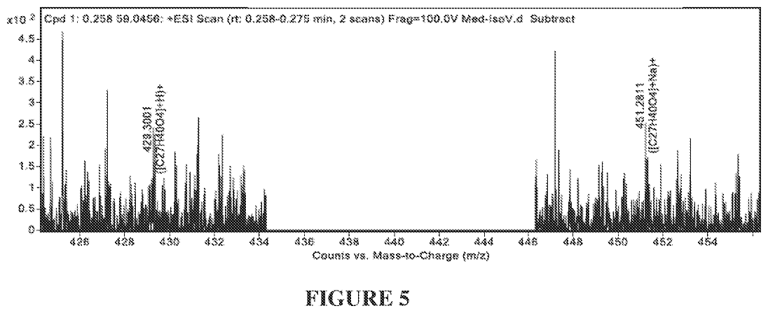

In round bottom flask isovaleric acid 2.8 mmol (0.28 ml) and trifluoroacetic anhydride 7 2 mmol (1.46 ml) were taken and stirred under ice cooled condition. To the reaction mixture 0.5 g of medroxyprogesterone was added in portions and then catalytic amount of p-toluenesulfonic acid (PTSA) was added (around 2 mg). The reaction mixture was stirred at room temperature and its progress was monitored via TLC using ethyl acetate and hexane (30:70 ratio) as solvent system. After 7 hours starting material was completely consumed and a new prominent spot appeared on TLC. Further, to the reaction mixture 50 ml of saturated sodium bicarbonate was added and extracted twice with ethyl acetate (100 ml). Combined ethyl acetate layer was first washed with ice cold water 2 ml), and then with brine water (100 ml). Ethyl acetate layer was dried over anhydrous sodium sulphate and finally evaporated on rotary evaporator to afford medroxyprogesterone 17-isovalerate. The HRMS spectrum of the medroxyprogesterone 17-isovalerate is shown in FIG. 5. The mass spectral data were as follows:

| m/z | Calc m/z | Diff(ppm) | z | Abund | Formula | Ion |
|---|---|---|---|---|---|---|
| 429.3001 | 429.2999 | −0.49 | 1 | 128.64 | C27H40O4 | (M + H)+ |
| 451.2811 | 451.2819 | 1.8 | 1 | 176.79 | C27H40O4 | (M + Na)+ |

### Example 4: Synthesis of medroxyprogesterone 17-cyclopentylacetate

In the same manner given in Example 3, treating medroxyprogesterone with 2-cyclopentylacetic acid (cyclopentylacetic acid) in the presence of p-toluenesulfonic acid (PTSA) catalyst at room temperature afforded medroxyprogesterone 17-cyclopentylacetate.

### Example 5: Synthesis of medroxyprogesterone 17-cyclopropanepropanoate

In the same manner as shown in Example 3, medroxyprogesterone was treated with 3-cyclopropylpropanoic acid (cyclopropanepropanoic acid) to give metroxyprogesterone 17-cyclopropanepropanoate.

### Example 6: Synthesis of medroxyprogesterone 17-isobutyrate

In the same manner given in Example 4, treating medroxyprogesterone with isobutyric acid in the presence of p-toluenesulfonic acid (PTSA) catalyst at room temperature afforded medroxyprogesterone 17-cyclopentylacetate.

I claim:

1. A compound of Formula Ia:

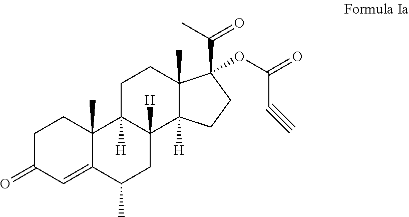

2. A compound of Formula Ib:

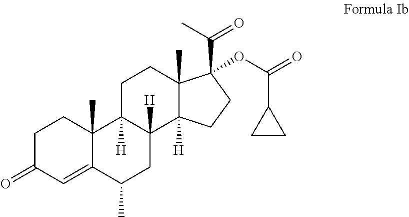

3. A compound of Formula Ic:

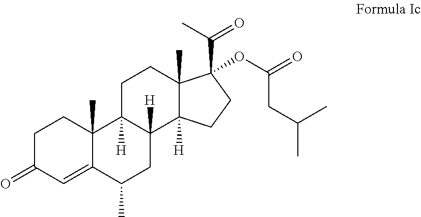

4. A compound of Formula Id:

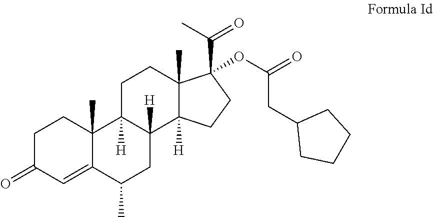

5. A compound of Formula Ie:

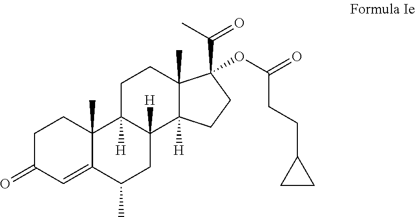

6. A compound of Formula If:

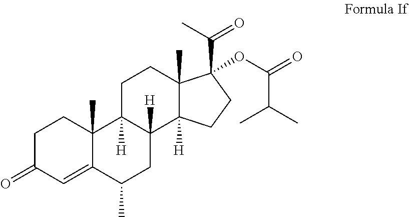

7. A pharmaceutical composition comprising a medroxyprogesterone ester and at least one pharmaceutically acceptable carrier, wherein the medroxyprogesterone ester is selected from the group consisting of:

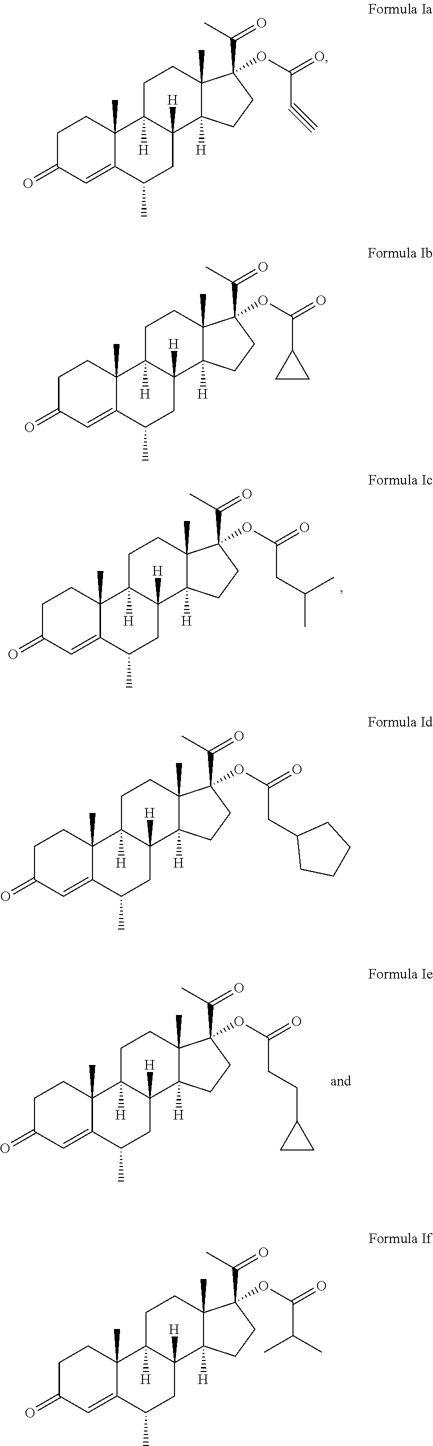

8. The pharmaceutical composition as claimed in claim 7, wherein the composition is formulated for parenteral administration.

9. The pharmaceutical composition as claimed in claim 7, wherein the composition is formulated for intramuscular or subcutaneous administration.

10. A method of providing contraception or treating hormone-dependent conditions in a mammal, the method comprising administering to the mammal a therapeutically effective amount of a medroxyprogesterone ester, which is a compound selected from the group consisting of: [chemical structure]
